(19) United States
(12) Patent Application Publication
(10) Pub. No.: US 2020/0375338 A1
(43) Pub. Date: Dec. 3, 2020

(54) COSMETIC ARTICLE PROVIDED WITH A MESH

(71) Applicant: CHANEL PARFUMS BEAUTE, Neuilly sur Seine (FR)

(72) Inventors: Hélène VILLECROZE, Pantin (FR); Alexandre LAUER, Pantin (FR); Frédéric MILESI, Neuilly sur Seine (FR); Caroline MENARD, Chamant (FR)

(73) Assignee: CHANEL PARFUMS BEAUTE, Neuilly sur Seine (FR)

(21) Appl. No.: 16/497,266

(22) PCT Filed: Mar. 26, 2018
(86) PCT No.: PCT/FR2018/050726

§ 371 Date: Sep. 24, 2019

(30) Foreign Application Priority Data
Mar. 31, 2017 (FR) 1752768

(51) Int. Cl. A45D 33/02; A45D 33/00

(52) U.S. Cl. CPC A45D 33/025; A61K 8/062; A45D 33/006

(57) ABSTRACT

A cosmetic article including a casing; a reservoir housed in the casing, including a thixotropic cosmetic composition and having an opening for dispensing the cosmetic composition; and a mesh covering the opening of the reservoir, configured to be impermeable to the cosmetic composition when it is not subject to a shear stress, and permeable to the cosmetic composition when it is subject to a shear stress.

The present invention relates to the field of cosmetics, in particular the fields of make-up, care, or fragrancing compositions.

Although not confined thereto, the invention has particularly beneficial applications in the more specific field of compositions for the face, such as foundation, complexion corrector, primer, concealer, blusher - sometimes referred to as blush, illuminator - sometimes referred to as highlighter, moisturizers, anti-wrinkle care, and sun protection care.

Cosmetic products, in particular make-up products, are traditionally products for use at home. With changing lifestyles, more and more consumers carry them around for use outside the home, but the products are not always suitable for such use. Outdoor—or portable—cosmetics must be compact and not be too bulky, to enable easy and optimal use of the product in all circumstances, in the same way as in the comfort of a bathroom.

Among the existing compact cosmetics, compact powders and cast products are the most common. However, such compositions are not suitable for all skin types or for all applications. In particular, compact powders are not compatible with care compositions, and cast products do not generally have good spreading properties due to the use of structuring waxes.

Fluid cosmetic compositions, on the other hand, are very much appreciated for their properties of spreading on the skin. They provide greater consumer satisfaction, especially in terms of the make-up result in the case of a make-up product, sensoriality, comfort, freshness and/or durable wear.

Among the compact packaging articles for fluid cosmetic compositions, there are especially impregnated sponges. These products, known under the name cushion, constitute a new category of products that are highly appreciated by consumers for their portability. A porous sponge, for example of the urethane type, is placed in a compact casing, and is impregnated with a fluid cosmetic composition, as described in document WO2012128589. The product is then removed by pressing the impregnated sponge using an applicator, for example a second, non-porous, sponge. However, the dose taken can vary greatly depending on the pressure exerted by the user, and can even lead to an overflow of the fluid composition outside the sponge. This type of implementation also has numerous technical constraints. It requires perfectly mastering the structural parameters of the sponge, especially its porosity and its hardness. In addition, such a cosmetic article does not allow complete return of the product to the consumer because of the absorption and adsorption of the product by the sponge. Thus, to compensate for the loss of product, it is necessary to increase the size of the sponge to increase the amount of product, which does not favor the portability of the device.

Devices are also known that comprise a net stretched over a reservoir, retaining a creamy fluid composition, as described in document KR101477583. In this case, the net must be sufficiently permeable to allow the cosmetic composition to pass through, but without being excessively permeable, since the composition could flow through the net that would no longer be fulfilling its function of retaining the composition. This document describes in particular an optimum viscosity range of between 20 and 40 Pa·s to avoid problems of leakage. Outside this viscosity range, the described device is no longer suitable. However, cosmetic compositions having good spreading properties are ideally below 10 Pa·s, preferably below 7 Pa·s, more preferentially between 1 Pa·s and 3 Pa·s. In addition, the net of the cosmetic article of this document is inserted between two circular guides that fit inside one another by virtue of lips and projections, this assembly then resting on the walls of the body of the reservoir. Two other closure elements are necessary to ensure leaktightness. This reservoir is formed of many parts that do not provide maximum leaktightness. The device of KR101477583 is therefore not suitable for a portable use and packaging of cosmetic compositions having good spreading properties.

Document EP2837306 describes a device combining the various approaches mentioned above: a mesh is associated with an absorbent material to dispense the cosmetic product. The absorbent material is typically a sponge and serves as a container for a liquid cosmetic composition. It also makes it possible to eject this same liquid through the screen when a force is applied vertically. This type of product has the disadvantages of sponges without the advantages of the use of a mesh, namely homogeneous dispensing, low packaging and full consumption of the product.

An object of the invention is therefore to provide a cosmetic article intended for portable use, enabling the packaging and use of a cosmetic composition having optimal properties, and especially good spreading. Such a cosmetic article must in particular be compact and protect the user from any risk of leakage or overflow during transportation of the device and use thereof.

To this end, according to the invention a cosmetic article is provided, comprising:

i. a casing,

ii. a reservoir housed in the casing, comprising a thixotropic cosmetic composition and having an opening for dispensing said cosmetic composition,

iii. a mesh covering said opening of the reservoir, configured to be impermeable to the cosmetic composition when the latter is not subject to a shear stress, and permeable to the cosmetic composition when the latter is subject to a shear stress.

The use of a thixotropic cosmetic composition in combination with a mesh makes it possible to combine a cosmetic composition having excellent spreading properties with portable use. This is because, during the collection and spreading thereof, for example using a sponge, the composition is subjected to a shear stress, which destructures it and makes it fluid, resulting in good spreading. The cosmetic composition can thus afford freshness and moisturization. In addition, this enables easy collection of the composition through the mesh: the consumer can precisely control the dose of the composition collected.

Before use and once the use is complete, the cosmetic composition becomes restructured and has a sufficiently high viscosity to not be able to pass through the mesh. This enables portable use of the cosmetic article. This is because the cosmetic composition cannot leak from the cosmetic article in its native state, which is a structured and viscous state. The cosmetic composition is also not able to overflow since the mesh is impermeable to the cosmetic composition when the latter is not subject to a shear stress, that is to say when it is in its native state.

The rheological behavior of the cosmetic composition thus enables great compatibility with portable casings provided with a mesh, while overcoming the constraints of the prior art described above. The cosmetic article according to the invention can be used without impregnated sponge, which makes it possible to significantly reduce the volume of the reservoir and prevent any loss of product. The absence of the risk of flow during the transport of the composition greatly reduces the design constraints for the packaging of the cosmetic article, especially the mesh.

The term mesh for the purposes of the invention denotes alternating fibers organized in a network. It may typically be a fabric, a net or else a metal grid. The fibers may be of any type, especially textile or metallic, but are preferably made of a polymeric material. The fibers may all be identical, or on the contrary be different. Preferably, the mesh comprises polyethylene terephthalate (PET) fibers and polyurethane fibers. A large proportion of polyurethane fibers may advantageously be used in order to give the mesh high elasticity.

Advantageously, the mesh is sufficiently elastically deformable to make it possible to reach the bottom of the reservoir. This makes it possible for the cosmetic composition to be entirely returned to the consumer, without loss of product due to the packaging means.

The organization of the fibers in a network can be of any type, but preferably they are knit-woven. A knit weave makes it possible to give the mesh a good rigidity while taking advantage of the elasticity of the elastic fibers that the mesh optionally contains. The organization of the fibers of the mesh defines spacings between the fibers. The term pitch will be used to refer to the mean dimension of these spacings. The pitch of the mesh and the dimensions of the largest spacings between the fibers will have a significant impact on the permeability of the mesh. Preferably, the mesh pitch is between 0.01 and 1 mm.

The mesh of the cosmetic article according to the invention is configured to be impermeable to the cosmetic composition when the latter is not subject to a shear stress, and permeable to the cosmetic composition when the latter is subject to a shear stress. In order to configure the mesh in this way, it will be possible to vary the pitch of the mesh, the size of the largest of these spacings, or else the material from which the fibers of the mesh are made. Indeed, a mesh made of hydrophobic fibers will for example be less permeable to hydrophilic compositions, and vice versa. These adjustments will not pose any particular difficulty for those skilled in the art. Particular cases will be illustrated in the exemplary embodiments of the invention, detailed below.

The cosmetic composition contained in the cosmetic article according to the invention preferably comprises an oil-in-water composition having an aqueous phase and a fatty phase, the aqueous phase comprising a thixotropic gelling agent. The use of oil-in-water emulsions makes it possible to obtain a formula that is very fresh upon application, which is highly appreciated by consumers.

The thixotropic gelling agent is preferably a nonionic block copolymer comprising at least one hydrophilic portion and a hydrophobic portion. Block copolymers are also known under the name associative polymers or block polymers.

Due to the combined presence of hydrophobic and hydrophilic portions, and although in the preferred embodiment of the invention the thixotropic agent is included in the aqueous phase of an oil-in-water emulsion, said thixotropic agent could also be included in the fatty phase of the emulsion or even at the interface between the phases, for example within a possible emulsifier, without departing from the scope of the present invention. Nonetheless, the fact that the thixotropic agent is included in the aqueous phase enables the different macromolecules to interact reversibly via their hydrophobic portions, which favors the formation of a gel with thixotropic properties.

The hydrophilic portions and the hydrophobic portions of this thixotropic agent are for example connected by at least one urethane bond.

Similarly, the nonionic block copolymer may advantageously be chosen from polyurethanes and derivatives thereof.

Urethane bonds have numerous advantages, especially that of being compatible with a cosmetic use. Urethane groups also have excellent stability and exceptional rigidity, which is reflected in very good mechanical properties of the thixotropic agent, including when the urethane groups are only present at the junction between the hydrophilic portions and the hydrophobic portions of the thixotropic agent.

In one advantageous embodiment, the hydrophilic portion of such a thixotropic agent is polyoxyethylene-based—it may for example be polyethylene glycol (PEG)—and/or the hydrophobic portion is hydrocarbon-based, for example hydrocarbon-based chains comprising between 6 and 40 carbon atoms. Polyoxyethylene-based polymers, and especially PEG, generally have a viscosity which decreases when the temperature increases. These highly hydrophilic polymers also have good stability to heat and dioxygen, which is particularly advantageous for portable cosmetic use. Indeed, the cosmetic article will be more regularly exposed to heat in the case of portable use than in the case of use in a bathroom. In particular, the cosmetic article might be placed in the pocket of a garment and warmed by proximity to the user's body. It might also be inadvertently left in direct sunlight.

Preferably, the nonionic block copolymer consists of a chain of three successive blocks, the first and the third block having hydrophilic properties and the second block having hydrophobic properties. For example, the copolymer may have a structure of A-B-A′ in which A and A′ represent the hydrophobic portions and B the hydrophilic portion. The portions A and A′ may be identical or different. The hydrophobic portions at each end can then assemble together, reversibly, and thus form a three-dimensional network responsible for the gelation of the composition and its thixotropic character. Such a copolymer is for example PEG-240/HDI COPOLYMER BIS-DECYLTETRADECETH-20 ETHER (INCI name) available under the reference Adeka Nol GT-700, or Adeka Nol GT-730 from Adeka. This polymer is used as a thixotropic agent in a preferred embodiment of the invention.

The content by weight of the thixotropic gelling agent relative to the total weight of the composition is between 0.01% and 20%, preferably between 0.5 and 10%, more preferably still from 1 to 5%. The content must be sufficient to give the composition the desired rheological properties, and may therefore vary depending on the thixotropic agent chosen.

The cosmetic composition must have a particular rheological behavior, favoring the packaging and dispensing of the composition by the cosmetic article according to the present invention, intended for portable use. In particular, the composition has a shear-thinning character: after the application of shearing, the viscosity of the composition decreases. “Thixotropic cosmetic composition” is intended to mean a cosmetic composition having at least a shear-thinning character. In addition, after a rest period, the composition partially or completely regenerates its viscosity, consistency and initial elasticity. The recovery in viscosity, consistency, and elasticity is delayed in time. The composition may advantageously have a shape memory behavior, that is to say that it can be deformed into a temporary configuration, under the effect of a mechanical stress for example, and then be restored to the original geometry after a certain amount of time after the stress has stopped. Such a composition is sometimes termed a weak gel. Macroscopically, this is reflected in the fact that when a user collects the composition from a container containing the composition, the surface becomes smooth and horizontal again, as it was initially, after a certain amount of time. This property not only makes it possible to keep the same aesthetic appearance as when the cosmetic article according to the present invention was first used, but it also enables the user to control the dose of composition collected at each new use, until the cosmetic composition is entirely exhausted.

Indeed, when the composition is returned uniformly behind the mesh, this enables the user to collect the same dose each time.

The cosmetic composition preferably has a plateau viscosity of greater than or equal to 2500 Pa·s, preferably greater than or equal to 2700 Pa·s.

The cosmetic composition preferably has a yield stress of less than or equal to 60 Pa, preferably less than or equal to 55 Pa. The yield stress corresponds to the minimum stress needed to cause the product to flow. This value provides information regarding the spreadability of the composition and its ability to be extruded through the mesh. A sufficiently low yield stress is required to facilitate collection.

The cosmetic composition preferably has a complex modulus G* of between 200 and 600 Pa, preferably between 300 and 500 Pa.

The composition preferably has a phase angle δ of between 7° and 20° , preferably between 7° and 10°.

The composition preferably has a viscosity at 100 s⁻¹ of less than 3 Pa·s, preferably of between 1 and 3 Pa·s. The viscosity at a shear rate of 100 s⁻¹ corresponds to a stress equivalent to that exerted at the moment of spreading on the skin.

These different properties are measured according to the following protocols.

The different viscosity of the composition contained in the article according to the invention is measured at 20° C. using the DHR2 rheometer from TA Instruments, fitted with a sanded plate-plate measuring body 40 mm in diameter. The measurements are carried out following a flow ramp. The composition is first brought to temperature at 20° C. ±1° C. for 180 seconds without any shearing being applied thereto. A stress sweep is then applied to the composition for 400 seconds, between 0.01 Pa and 1000 Pa.

The change in the viscosity 11 is represented graphically as a function of the shear gradient applied 6. The measurement points are logarithmically distributed at an amount of 50 points per decade. The plateau viscosity corresponds to the viscosity at rest. Graphically, these are the first points forming a plateau at low shear.

The state of structure of the compositions is studied via an oscillation sweep with a stress ramp using the DHR-2 rheometer from TA Instruments. The device is fitted with a “sanded” plate-plate measuring body 40 mm in diameter. The composition is brought to temperature at 20° C. for 60 seconds without any stress being applied thereto. A sweep of increasing stresses is then applied, between 0.01 and 1000 Pa, at a fixed frequency of 1 Hz. The change in the complex modulus G* (in Pa) as a function of the oscillatory stress applied (in Pa) is thus measured.

This method makes it possible to determine the following parameters:
the complex shear modulus G* (in Pa) of the composition, which makes it possible to characterize the consistency of the composition and its viscoelastic properties. It corresponds to the ratio of the stress imposed to the strain measured.
the phase angle δ (in degrees) which provides information regarding the more or less solid or liquid behavior of the composition. The phase angle of a product is between 0 and 90° (at 0° reference is made to a perfect solid, and at 90° reference is made to a perfect liquid).

The values of G* and δ retained are the “plateau” values, that is to say corresponding to the stress zone for which there is less than 10% variation in these two values.

The cosmetic composition contained in the cosmetic article according to the invention may comprise other components.

Composition—Aqueous Phase

The cosmetic composition comprises one or more aqueous phases. “Aqueous phase” is intended to mean a phase comprising water and generally any molecule in the state dissolved in water in the composition.

The aqueous phase of said compositions contains water and in general other water-soluble or water-miscible solvents. The water-soluble or water-miscible solvents include monoalcohols with a short chain, for example a C1-C4 chain, such as ethanol or isopropanol; humectants such as hyaluronic acid salts such as sodium hyaluronate, diols or polyols such as ethylene glycol, 1,2-propylene glycol, 1,3-butylene glycol, caprylyl glycol, hexylene glycol, diethylene glycol, dipropylene glycol, and mixtures thereof. Mention may also be made of polysaccharide derivatives obtained by bacterial fermentation, such as the compound known under the INCI name Biosaccharide gum-1.

The composition may further comprise an emulsifying system, or emulsifier. The emulsifying system may consist of a surfactant having an HLB (hydrophilic/lipophilic balance) of less than 7. Preferably, the emulsifying surfactant is then chosen from fatty acid esters of polyols, such as mono, di, tri or sesquioleates or stearates of sorbitol or of glycerol, laurates of glycerol or of polyethylene glycol; silicone surfactants such as dimethicone copolyols or alkyl dimethicone copolyols bearing an alkyl chain that is pendent or at the end of the silicone backbone, having for example from 6 to 22 carbon atoms, such as PEG-10 dimethicone; polymers of polyoxyalkylenated glycol fatty acid ester type, alkyl or alkoxy dimethicone copolyols bearing an alkyl or alkoxy chain that is pendent or at the end of the silicone backbone, having for example from 6 to 22 carbon atoms, and mixtures thereof.

The composition may also comprise at least one gelling agent of the aqueous phase that is different from the abovementioned gelling agent, intended to confer the abovementioned rheological properties. The main hydrophilic gelling agents that can be used are crosslinked polymeric gelling agents and natural polymers.

Such crosslinked polymeric gelling agents are for example carboxyvinyl polymers, such as the products sold under the name Carbopol (INC1 name: carbomer) by Novéon, polyacrylamides, polymers derived from 2-acrylamido-2-methylpropanesulfonic acid (AMPS), such as the product sold by Clariant under the name Hostacerin AMPS, and crosslinked anionic copolymers of acrylamide and of AMPS, such as the product sold under the name SEPIGEL 305 or SEPINOV EMT 10 by SEPPIC.

The natural polymers that may be used are for example gellan gum, xanthan gum, and guar gum, or else cellulose-based derivatives, starches and alginates, and mixtures thereof.

These natural polymers can be used alone or in combination with the crosslinked polymeric gelling agents.

Composition—Oils

In addition, the composition preferably comprises 0.01% to 60% by weight relative to the total weight of the composition, more preferentially between 0.5% and 50%, even more preferentially between 1% and 30% of oils.

The oils that can be used may preferably be chosen from hydrocarbon-based oils, silicone oils, or mixtures thereof

For the purposes of the present invention, the term “oil” is intended to mean a compound that is liquid at room temperature (25° C.) and atmospheric pressure, and that, when it is introduced at an amount of at least 1% by weight into water at 25° C., is not at all soluble in water, or is soluble to an extent of less than 10% by weight, relative to the weight of oil introduced into the water. The oils that can be used in the present invention may be volatile or non-volatile. The oils may be of plant, mineral or synthetic origin.

Non-volatile oils of plant origin comprise in particular castor oil, sweet almond oil, sunflower oil, rice bran oil, macadamia nut oil, olive oil, wheatgerm oil, groundnut oil, sea buckthorn oil, borage oil, palm oil, camelia oil, gardenia oil and mixtures thereof.

The non-volatile oils of mineral origin comprise in particular paraffin oil and isoparaffin.

The non-volatile oils of synthetic origin comprise in particular hydrocarbon-based oils such as (poly)ethers or (poly)esters, in particular (poly)esters of C6 to C20 fatty acids and of C6 to C20 fatty alcohols which may advantageously be branched, such as diisostearyl malate, dicaprylyl carbonate, isononyl isononanoate; plant oils or derivatives thereof, such as hydrogenated castor oil; branched and/or unsaturated fatty acids; branched and/or unsaturated fatty alcohols; silicone oils such as linear polydimethylsiloxanes (INCI name: Dimethicone) which may optionally be phenylenated, cyclic polydimethylsiloxanes; and mixtures thereof.

By way of example, the composition contained in the article according to the invention may typically contain one or more non-volatile oils chosen from hydrocarbon-based oils such as squalane, polybutene, hydrogenated polyisobutene, or hydrogenated polydecene; phenyl silicone oils such as those that can be identified by the INCI names “phenyl trimethicone”, “phenylpropyldimethylsiloxysilicate” or “trimethyl pentaphenyl trisiloxane”, and fluoro silicone oils.

The composition contained in the cosmetic article according to the invention may also comprise at least one oil that is volatile at room temperature. For the purposes of the invention, the term “volatile oil” is intended to mean an oil capable of evaporating on contact with the skin or the keratin fiber in less than one hour. The volatile oils of the invention are volatile cosmetic oils that are liquid at room temperature and that have a non-zero vapor pressure at room temperature and atmospheric pressure. The volatile oils that may be used in the context of the invention may be silicone or hydrocarbon-based.

Examples of suitable volatile oils comprise volatile linear alkanes such as those described in document FR2933865 which is incorporated herein by way of reference. Examples of suitable volatile linear alkanes are C9-C17, in particular C10-C14, alkanes, such as the mixture of undecane and tridecane available under the trade name Cetiol® Ultimate from BASF or the C15-C19 alkanes available under the trade name Emogreen® L15 from SEPPIC, or C12-C14 alkanes available under the trade name Vegelight® 1214 LC from Biosynthis.

Examples of suitable silicone volatile oils include linear, branched or cyclic silicone oils, in particular having a viscosity of less than 10 cSt, and in particular having between 2 and 10 silicon atoms. Particular non-limiting examples include octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane, dodecamethylpentasiloxane, the compounds with the INCI names corresponding to Methyl Trimethicone, and mixtures thereof

Of course, the composition contained in the article according to the invention may comprise mixtures of the oils mentioned above.

Composition—Film Forming Agents

The composition contained in the article according to the invention may also comprise at least one film-forming component, in particular capable of imparting durable wear and/or non-transfer properties to the composition.

The film-forming agent is generally a polymer. This film-forming polymer may be a silicone polymer optionally modified by urethane or fluorine or acrylate, such as the silicone (meth)acrylates sold by Jéeen under the name Jéesil PS (which include PS-VH, PS-VHLV, PS-CM, PS-CMLV and PS-DMLV), or the polymers sold by Shin Etsu under the trade names KP-545, KP-561 and KP-562, or the polymers sold by Dow Corning under the trade names Dow Corning® FA 4003 DM, Dow Corning® FA 4002 ID and Dow Corning® FA 4001 CM. Other examples of film-forming polymers are silicone resins and in particular MQ resins such as trimethylsiloxysilicates sold by Wacker under the name Belsil TMS 803, and MT resins such as silsesquioxane derivatives and in particular the polymethylsilsesquioxanes sold in particular by Shin Etsu, and also the polypropylsilsesquioxane sold by Dow Corning under the trade name Dow Corning® 670 or the phenylpropyl polysilsesquioxane sold by Wacker under the trade name Belsil SPR45VP. Another example consists of the fluorosilicone polymers identified by the INCI name trifluoropropyldimethylsiloxy triethylsiloxysilicate, such as that sold by General Electric under the trade name XS66-B8226. Use may also be made, as film-forming polymers, of bioadhesive polymers obtained for example by polycondensation of dimethiconol and of MQ silicate resin in a solvent such as heptane, which are in particular sold by Dow Corning under the trade names Dow Corning® BIO-PSA 7-4560 Silicone Adhesive, Dow Corning® 7-4405 low tack and Dow Corning® 7-4505 high tack. Other examples of film-forming polymers are cyclic polyolefins such as polycyclopentadiene, in particular sold by Kobo under the trade name Koboguard 5400, or else polydicyclopentadiene. Other further examples of film-forming agents consist of copolymers of vinylpyrrolidone (VP) and/or of linear olefins, such as VP/hexadecene and VP/eicosene polymers, including Antaron V216 and Antaron V220 from ISP or else ethylene/vinyl acetate copolymers, such as AC 400 from Baerlocher. Other film-forming polymers able to be used in this invention are polyacrylates such as the poly(ethyl acrylate) sold in particular by Creations Couleurs under the trade name Creasil 7 ID.

Composition—Ultraviolet Screening Agents

The cosmetic composition may also comprise 0% to 50% by weight of at least one ultraviolet (UV) screening agent, preferably from 0% to 30%, more preferentially from 0% to 20% by total weight of the composition. In particular, the composition contained in the article according to the invention may comprise at least one organic or mineral sunscreen or a mixture of the two.

By way of illustration of organic UV screening agents and in a nonlimiting manner, mention may be made of:
anthranilates, in particular menthyl anthranilate;
benzophenones, in particular benzophenone-1, benzophenone-3 or oxybenzone, benzophenone-5, benzophenone-6, benzophenone-8, benzophenone-9, benzophenone-12, and preferentially benzophenone-2 (oxybenzone) or benzophenone-4 (Uvinul MS400 available from BASF);
benzylidenecamphors, in particular 3-benzylidenecamphor, benzylidenecamphorsulfonic acid, camphor benzalkonium methosulfate, polyacrylamidomethylbenzylidenecamphor, terephthalylidenedicamphorsulfonic acid, and preferentially 4-methylbenzylidenecamphor (Eusolex 6300® available from Merck);
benzimidazoles, in particular benzimidazilate (Neo Heliopan AP® available from Haarmann and Reimer), or phenylbenzimidazole sulfonic acid (Parsol HS® available from DSM);
benzotriazoles, in particular drometrizole trisiloxane, or methylene bis-benzotriazolyl tetramethylbutylphenol (Tinosorb M® available from Ciba);
cinnamates, in particular cinoxate, DEA methoxycinnamate, diisopropyl methylcinnamate, glyceryl ethylhexanoate dimethoxycinnamate, isopropyl methoxycinnamate, isoamyl cinnamate, Kaempferia galanga root extract (Tego Galanga from Evonik containing 98% ethyl-p-methoxycinnamate) and preferentially octylmethoxycinnamate (Parsol MCX® available from Hoffmann La Roche);
diphenylacrylates, in particular ethocrylene (Uvinul N35® available from BASF), or octocrylene (Uvinul 539® available from BASF) or ethylhexyl methoxycrylene (Solastay available from Hallstar);
dibenzoylmethanes, in particular butyl methoxydibenzoylmethane (Parsol 1789®); imidazolines, in particular ethylhexyl dimethoxybenzylidene dioxoimidazoline;
PABAs, in particular ethyl dihydroxypropyl PABA, ethylhexyldimethyl PABA, glyceryl PABA, PABA, PEG-25 PABA, or ethyl PABA (benzocaine);

1triazines, in particular anisotriazine (Tinosorb S® available from Ciba) or diethylhexylbutamidotriazone (Uvasorb HEB® available from 3V Sigma), ethylhexyltriazone (Uvinul T1500 available from BASF), tris-biphenyl triazine (Tinosorb 2AB available from BASF);
benzoates, in particular N-hexyl 2-(4-diethylamino-2-hydroxybenzoyl)benzoate (Uvinul A+available from BASF) or as a mixture with octyl methoxycinnamate (Uvinul A+B available from BASF);
benzalmalonates, in particular polysilicone-15 (Parsol SLX available from DSM);
benzoxazoles, in particular 2,4-bis[4-[5-(1,1-dimethylpropyl)benzoxazol-2-yl]phenylimino]-6-[(2-ethylhexyl)imino]-1,3,5-triazine (Uvasorb K2A available from 3V Sigma);
salicylates, in particular dipropylene glycol salicylate, ethylhexyl salicylate, homosalate, butyloctyl salicylate (Hallbrite BHB available from Hallstar) or TEA salicylate.

The inorganic UV screening agents used are, for example, metal oxide particles having an average elementary particle size of less than or equal to 300 nm, preferably less than or equal to 100 nm.

They may in particular be chosen from titanium oxide, zinc oxide, iron oxide, zirconium oxide, cerium oxide or mixtures thereof. The titanium oxides may be in a crystalline form of rutile and/or anatase type, and/or in an amorphous or substantially amorphous form.

The coated titanium oxide pigments may be coated:
with silica (Sunveil from Ikeda);
with silica and iron oxide (Sunveil F from Ikeda);
with silica and polyglyceryl-10 stearate (Cosmeserve WP-40W from Iwase Cosfa);
with silica and alumina (Microtitanium Dioxide MT500 SA and Microtitanium Dioxide MT 100 SA from Tayca, Tioveil from Tioxide);
with alumina (Tipaque TTO-55 (B) and Tipaque TTO-55 (A) from Ishihara, and UVT 14/4 from Kemira);
with alumina-treated rutile TiO₂, and with glycerol-coated silica (UV Titan M212 from Kemira);
with alumina-treated rutile TiO₂ and with dimethicone (UV Titan M195 from Kemira);
with alumina and with aluminum stearate (Microtitanium Dioxide MT 100 T, MT 100 TV, MT 100 TX, MT 100 Z, MT-01 from Tayca, Solaveil CT-10 W and Solaveil CT 100 from Uniqema and Eusolex T-AVO from Merck);
with silica, with alumina and with alginic acid (MT-100 AQ from Tayca);
with alumina and with aluminum laurate (Microtitanium Dioxide MT 100 S from Tayca);
with alumina, with methicone and with polyhydroxystearic acid (INP60T7 from Kobo);
with iron oxide and with iron stearate (Microtitanium Dioxide MT 100 F from Tayca);
with zinc oxide and with zinc stearate (BR 351 from Tayca);
with silica and with alumina and treated with a silicone (Microtitanium Dioxide MT 600 SAS, Microtitanium Dioxide MT 500 SAS or Microtitanium Dioxide MT 100 SAS from Tayca);
with silica, with alumina, with aluminum stearate and treated with a silicone (STT-30-DS from Titan Kogyo);
with alumina and treated with a silicone (Tipaque TTO-55 (S) from Ishihara, or UV Titan M 262 from Kemira);
with triethanolamine (STT-65-S from Titan Kogyo);
with stearic acid (Tipaque TTO-55 (C) from Ishihara);
with sodium hexametaphosphate (Microtitanium Dioxide MT 150 W from Tayca);
with octyltrimethylsilane-treated TiO₂ (T 805 from Degussa Silices);
with polydimethylsiloxane-treated TiO₂ (70250 Cardre UF TiO2S13 by Cardre);
with polydimethylhydrosiloxane-treated anatase/rutile TiO₂ (Microtitanium Dioxide USP Grade Hydrophobic by Color Techniques);
with rutile TiO₂ treated with alumina, stearic acid (UV Titan M160 by Kemira);
with TiO₂ treated with alumina hydroxide, stearic acid and triethoxycaprylylsilane (ALT-T-400 by Maprecos or Titanium Dioxide & Aluminum hydroxide & Stearic acid (ST-705SA /Titan Kogyo));
with manganese-doped TiO₂ (OPT1-PW from Croda).

The uncoated titanium oxide pigments are for example:
Microtitanium Dioxide MT 500 B or Microtitanium Dioxide MT600 B by Tayca;
P 25 by Degussa;
PW transparent titanium oxide by Wacker;
UFTR by Miyoshi Kasei;
ITS by Tomen;

and Tioveil AQ by Tioxide.

The uncoated zinc oxide pigments are for example:
Z-cote by Sunsmart;
Nanox by Elementis;
Nanogard WCD 2025 by Nanophase Technologies.

The coated zinc oxide pigments are for example:
zinc oxide CS-5 by Toshibi (ZnO coated with polymethylhydrosiloxane);
Nanogard Zinc Oxide FN by Nanophase Technologies (as a 40% dispersion in Finsolv TN, C12-C15 alkyl benzoates);
Daitopersion ZN-30 and Daitopersion Zn-50 by Daito (dispersions in cyclopolymethylsiloxane/oxyethylenated polydimethylsiloxane, containing 30% or 50% of zinc nano oxides coated with silica and polymethylhydrosiloxanes);
NFD Ultrafine ZnO by Daikin (ZnO coated with perfluoroalkyl phosphate and copolymer based on perfluoroalkylethyl as a dispersion in cyclopentasiloxane);
SPD-Z1 by Shin-Etsu (ZnO coated with silicone-grafted acrylic polymer, dispersed in cyclodimethylsiloxane);
Escalol Z100 by ISP (alumina-treated ZnO dispersed in the ethylhexyl methoxycinnamate/PVP-hexadecene copolymer/methicone mixture);
Fuji ZnO-SMS-10 by Fuji Pigment (ZnO coated with silica and polymethylsilsesquioxane);
Nanox Gel TN by Elementis (ZnO dispersed at 55% in C12-C15 alkyl benzoate with polycondensate of hydroxystearic acid);
OTS-5 MZ-500 by Daito (ZnO dispersed in triethoxycaprylylsilane).

The uncoated cerium oxide pigments may for example be those sold under the name Colloidal Cerium Oxide by Rhone Poulenc.

Composition - pigments

The composition contained in the article according to the invention may comprise from 0 to 50% by weight of at least one pigment.

“Pigments” should be understood to mean white or colored, mineral and/or organic particles that are insoluble in an aqueous solution and that are intended to color and/or opacify the composition and/or the deposit produced from the composition.

The pigments may be chosen from mineral pigments, organic lakes, nacres, optical-effect pigments, such as reflective particles or interference pigments.

The mineral pigments may be chosen from metal oxide pigments, chromium oxides, iron oxides, titanium dioxide, zinc oxides, cerium oxides, zirconium oxides, manganese violet, Prussian blue, ultramarine blue, ferric blue, chromium hydrate and mixtures thereof.

The composition contained in the article according to the invention may also comprise a particular pigmentary titanium oxide coated with aluminum hydroxide and stearic acid, sold by Titan Kogyo under the name ST 705 SA. This pigment, already mentioned in the paragraph relating to ultraviolet screening agents, has a strong transmittance of rays of red color while at the same time blocking the other rays of the visible spectrum, and increases the sun protection factor.

The cosmetic composition may also comprise 0 to 50%, preferably 0 to 30% by weight, relative to the total weight of the composition, of at least one interference pigment. Interference pigments are pigments capable of generating color by an interference phenomenon. They may consist of a substrate, for example natural mica, silica, synthetic mica, titanium dioxide or glass, around which are deposited one or more layers of a material with a different refractive index, such as titanium dioxide, iron oxide or silica for example.

By way of illustration of interference pigments that may be introduced into the composition, mention may be made of the gold-colored interference pigments in particular sold by Engelhard under the name Brilliant gold 212G (Timica), Gold 222C (Cloisonne), Sparkle gold (Timica), Gold 4504 (Chromalite) and Monarch gold 233X (Cloisonne); the bronze interference pigments in particular sold by Merck under the name Bronze fine (17384) (Colorona) and Bronze (17353) (Colorona) and by Engelhard under the name Super bronze (Cloisonne); the orange interference pigments in particular sold by Engelhard under the name Orange 363C (Cloisonne) and Orange MCR 101 (Cosmica) and by Merck under the name Passion orange (Colorona) and Matte orange (17449) (Microna); the brown-tinted interference pigments in particular sold by Engelhard under the name Nu-antique copper 340XB (Cloisonne) and Brown CL4509 (Chromalite); the copper-tinted interference pigments in particular sold by Engelhard under the name Copper 340A (Timica); the red-tinted interference pigments in particular sold by Merck under the name Sienna fine (17386) (Colorona); the yellow-tinted interference pigments in particular sold by Engelhard under the name Yellow (4502) (Chromalite); the gold-tinted red-colored interference pigments in particular sold by Engelhard under the name Sunstone G012 (Gemtone); the pink interference pigments in particular sold by Engelhard under the name Tan opale G005 (Gemtone); the gold-tinted black interference pigments in particular sold by Engelhard under the name Nu-antique bronze 240 AB (Timica), the blue interference pigments in particular sold by Merck under the name Matte blue (17433) (Microna), the silvery-tinted white interference pigments in particular sold by Merck under the name Xirona Silver and the golden-green pink-orange interference pigments sold in particular by Merck under the name Indian summer (Xirona) and mixtures thereof.

The composition may also comprise organic lakes. Organic lakes are organic pigments formed of a dye attached to a substrate. They may for example be chosen from:
cochineal carmine;
organic pigments of azo dyes, anthraquinone dyes, indigoid dyes, xanthene dyes, pyrene dyes, quinoline dyes, triphenylmethane dyes or fluoran dyes. Among the organic pigments, mention may in particular by made of those known under the following names: D&C Blue no. 4, D&C Brown no. 1, D&C Green no. 5, D&C Green no. 6, D&C Orange no. 4, D&C Orange no. 5, D&C Orange no. 10, D&C Orange no. 11, D&C Red no. 6, D&C Red no. 7, D&C Red no. 17, D&C Red no. 21, D&C Red no. 22, D&C Red no. 27, D&C Red no. 28, D&C Red no. 30, D&C Red no. 31, D&C Red no. 33, D&C Red no. 34, D&C Red no. 36, D&C Violet no. 2, D&C Yellow no. 7, D&C Yellow no. 8, D&C Yellow no. 10, D&C Yellow no. 11, FD&C Blue no. 1, FD&C Green no. 3, FD&C Red no. 40, FD&C Yellow no. 5, FD&C Yellow no. 6.
insoluble sodium, potassium, calcium, barium, aluminum, zirconium, strontium or titanium salts of acid dyes such as azo, anthraquinone, indigoid, xanthene, pyrene, quinoline, triphenylmethane or fluoran dyes, these dyes possibly comprising at least one carboxylic or sulfonic acid group.

The organic lakes may also be supported by an organic support such as rosin or aluminum benzoate, for example.

Mention may also be made of liposoluble dyes such as, for example, Sudan Red, DC Red 17, DC Green 6, beta-carotene, soybean oil, Sudan brown, DC Yellow 11, DC Violet 2, DC orange 5 or quinolein yellow.

The chemical materials corresponding to each of the abovementioned organic colorants are mentioned in the book “International Cosmetic Ingredient Dictionary and Handbook”, Edition 1997, pages 371 to 386 and 524 to 528, published by “The Cosmetic, Toiletry, and Fragrance Association”, the content of which is incorporated into the present application by way of reference.

Composition—Fillers

The cosmetic composition may also comprise at least one filler, that is to say at least one non-colored insoluble particle. Said filler may be of mineral or organic nature, of any form. Fillers have the effect of modifying the rheology of the composition, the texture, the sensoriality and/or the make-up result produced by the cosmetic composition by conferring additional properties such as mattness, uniformity, coverage and/or stability.

By way of example, mention may be made of talc, mica, silica, hollow silica microspheres, calcium carbonate, magnesium carbonate, boron nitride, glass or ceramic microspheres, silica and titanium dioxide composite powders, kaolin.

As examples of organic fillers, mention may be made of polyamide powders, polyethylene powders, polymethyl methacrylate powders, polytetrafluoroethylene (Teflon) powders, silicone resin powders (for example Tospearl by Toshiba), natural or synthetic micronized waxes, metal soaps derived from carboxylic acids having 8 to 22 carbon atoms, solid elastomeric crosslinked organopolysiloxane particles such as that sold under the trade reference Dow Corning EP-9215 Cosmetic Powder by Dow Corning, solid elastomeric crosslinked organopolysiloxane coated with silicone resin, in particular coated with a silsesquioxane resin such as those sold under the trade references KSP-101, KSP-102, KSP-103, KSP-104, KSP-105 by Shin Etsu, and mixtures thereof.

All the compounds in the form of solid particles may have undergone a surface treatment, in particular in order to make them hydrophobic or hydrophilic. The pigments, the mineral screening agents and/or the fillers may be coated after having undergone one or more surface treatments.

The hydrophobic treatment agent may be chosen from silicones, such as methicones, dimethicones, perfluoroalkylsilanes; fatty acids, such as stearic acid; metal soaps, such as aluminum dimyristate, the aluminum salt of hydrogenated tallow glutamate, perfluoroalkyl phosphates, perfluoroalkyl silanes, perfluoroalkyl silazanes, poly(hexafluoropropylene oxide)s, polyorganosiloxanes comprising perfluoroalkyl perfluoropolyether groups, amino acids; N-acylamino acids or salts thereof; lecithin, isopropyl triisostearyl titanate, and mixtures thereof.

The N-acylamino acids may comprise an acyl group having from 8 to 22 carbon atoms, such as for example a 2-ethylhexanoyl, caproyl, lauroyl, myristoyl, palmitoyl, stearoyl or cocoyl group. The salts of these compounds may be the aluminum, magnesium, calcium, zirconium, zinc, sodium or potassium salts. The amino acid may for example be lysine, glutamic acid or alanine.

The term “alkyl” mentioned in the compounds mentioned above denotes in particular an alkyl group having from 1 to 30 carbon atoms, preferably having from 5 to 16 carbon atoms.

Composition—Fatty Phase Gelling Agents

The composition contained in the article according to the invention may comprise at least one fatty-phase gelling agent and/or thickener in addition to the thixotropic agent contained in the aqueous phase.

Among the fatty-phase gelling agents that can be used in the context of the invention, mention may in particular be made of organopolysiloxane elastomers (also known as silicone elastomers) which may for example be in the form of particles carried in at least one silicone or hydrocarbon-based non-volatile oil, thus forming a gel. This is more particularly a crosslinked silicone elastomer. The elastomer present in the composition according to the invention may be chosen from non-emulsifying or emulsifying elastomers.

The term “emulsifying organopolysiloxane elastomer” is intended to mean an organopolysiloxane elastomer comprising at least one hydrophilic chain, such as polyoxyalkylenated (polyoxyethylenated, polyoxypropylenated) organopolysiloxane elastomers and polyglycerolated silicone elastomers. As polyoxyalkylenated organopolysiloxane elastomers, use may be made of those sold under the names “KSG 21”, “KSG-20”, “KSG-30”, “KSG-31”, “KSG-33”, “KSG-210”, “KSG-310”, “KSG-330”, “KSG-340” by Shin Etsu, “DC9010”, “DC9011” by Dow Corning. As polyglycerolated organopolysiloxane elastomers, use may be made of those sold under the names “KSG-710”, “KSG-810”, “KSG-820”, “KSG-830”, “KSG-840” by Shin Etsu.

As non-emulsifying elastomers, use may for example be made of those sold under the names “DC 9040”, “DC 9041”, “DC 9509”, “DC 9505” by Dow Corning; “KSG-6”, “KSG-15”, “KSG-16”, “KSG-18”, “KSG-41”, “KSG-42”, “KSG-43”, “KSG-44” by Shin Etsu; Gransil SR SCYC gel, Gransil SR DMF 10 gel, Gransil SR DC556 gel from Gransil RPS of Grant Industries; 1229-02-167, 1229-02-168 and “SFE 839” from General Electric.

The fatty-phase gelling agents that can be used in the composition contained in the article according to the invention may be clays that have been modified to make them lipophilic. Mention may be made of hydrophobically modified montmorillonite clays, such as hydrophobically modified bentonites or hectorites. Mention may for example be made of the product Stearalkonium Bentonite (INCI name) (reaction product of bentonite and of the quaternary ammonium stearalkonium chloride) such as the commercial product sold under the name Tixogel MP 250 by Sud Chemie Rheologicals, United Catalysts Inc or the product Disteardimonium Hectorite (INCI name) (reaction product of hectorite and of distearyldimonium chloride) sold under the name Bentone 38 or Bentone Gel by Elementis Specialities.

Composition—Customary Cosmetic Ingredients

The cosmetic composition may also comprise additional customary cosmetic ingredients chosen in particular from antioxidants, fragrances, preservatives, neutralizing agents, vitamins, moisturizing agents, self-tanning compounds, antiwrinkle active agents, hydrophilic or lipophilic active agents, free-radical scavengers, deodorizing agents, sequestrants, and mixtures thereof.

The invention may be better understood using the nonlimiting exemplary embodiment described below, and with reference to the attached drawing, in which:

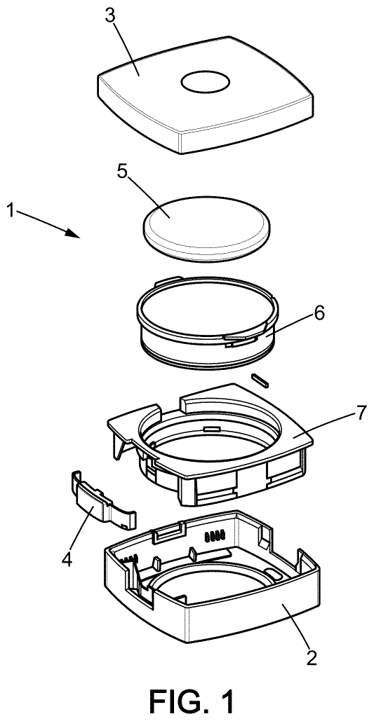

FIG. 1 shows an exploded view of an exemplary embodiment of the cosmetic article according to the invention,

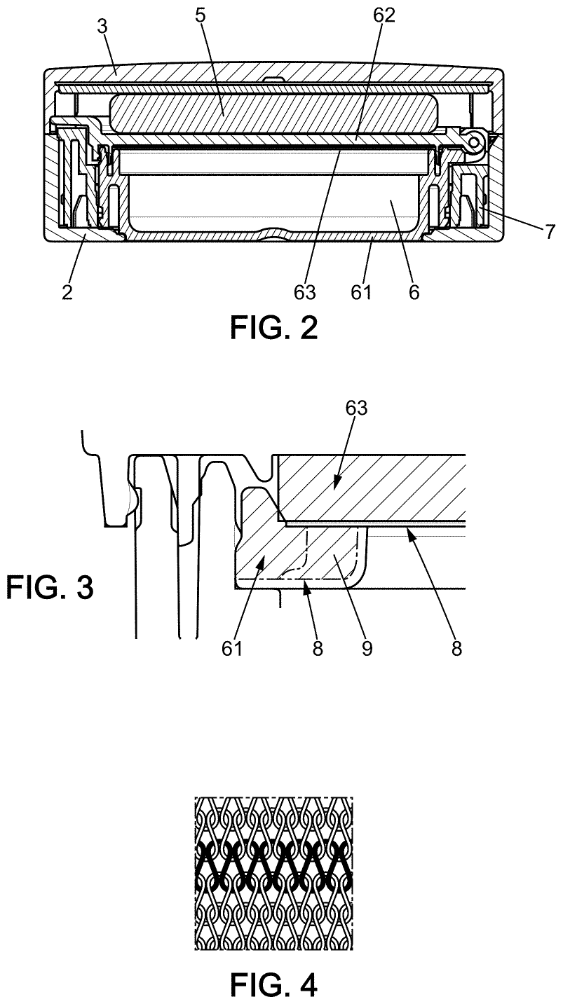

FIG. 2 shows a sectional view of the cosmetic article of FIG. 1,

FIG. 3 is a more precise sectional view of a detail of FIG. 2, and

FIG. 4 shows a preferred knit weave for the mesh of the cosmetic article according to the invention.

The cosmetic article 1 illustrated in FIGS. 1 to 3 comprising a casing for cosmetic product, comprising a main body in which a refillable and removable container 6 may be placed.

The casing is made of polypropylene or ABS. It is composed of a bottom 2 serving as housing for the refill container, and a cover 3 articulated to rotate on the bottom 2, the opening of which is subject to the actuation of a push-button 4. A puff 5 or an applicator sponge is positioned between the refillable container 6 and the cover 3 when the casing is in the closed position.

In order to arrange the housing, the bottom is fitted with a mounting plate 7, the walls of which are fitted with projections.

The refillable container 6 comprises a body 61 serving as reservoir for the cosmetic product, comprising a bottom, walls and an opening at the top making it possible to dispense the cosmetic product. It comprises at its base a groove which enables the container to fit inside the projections of the wall of the mounting plate. In this way, the container is put in place and blocked inside the casing by means of a snap-fastening mechanism.

The container further comprises a cover 62, articulated to rotate on the body 2 and associated with a closing mechanism for holding the cover closed against the body. This mechanism is equipped with three projections, being introduced into slots in the body to produce a snap-fastening, which ensures the leaktightness of the device after closing the cover on the body of the container. The cover may optionally be fitted with a seal 63 made of polyethylene terephthalate PET, adhesively bonded to the inner face of the cover 62.

A mesh 8 extends above the opening of the body, stretched and overmolded to a ring 9 which fits into a notch on the body 61 of the container 6. The overmolding may have been performed by any technique, especially by ultrasound. The ring 9 is preferably made of ABS; it has an outer wall, an inner wall, an upper surface and a lower surface. It comprises a notch on its outer wall, configured to receive the projection of the body of the container so as to allow the body 61 and the ring 9 to fit together. A planar surface is formed on the upper surface, extending from the inner edge of the ring. This surface may for example accommodate the seal 63. The mesh 8 is stretched at this planar surface.

The mesh 8 is configured such that the dispensing of the product contained in the body of the container can be effected through it. The mesh is composed of a majority of polyurethane fibers and a minority of PET fibers. These fibers are woven according to a knit weave illustrated in FIG. 4. This gives it sufficient elasticity to make it possible to reach the bottom and the corners of the body of the container.

The reservoir contains a thixotropic cosmetic composition. This composition has, for example, the formulation of composition 1 or composition 2 of table 1 below.

In table 1, the amounts are expressed as percentage by weight relative to the total weight of the composition.

TABLE 1
Formulations of compositions 1 to 4

| | Composition | Composition | Composition | Composition |
|---|---|---|---|---|
| Class of ingredients/INCI name | 1 | 2 | 3 | 4 |
| PEG-10 DIMETHICONE | — | — | — | 6 |
| DIMETHICONE & | — | — | — | 2 |
| TRIMETHYLSILOXYSILICATE/ | | | | |
| DIMETHICONOL CROSSPOLYMER | | | | |
| METHYL TRIMETHICONE | — | — | — | 7 |
| CYCLOPENTASILOXANE | — | — | — | 11.15 |
| DISTEARDIMONIUM HECTORITE | — | — | — | 0.15 |
| ETHYLHEXYL | 6.8 | 6.8 | — | 3 |
| METHOXYCINNAMATE & BHT | | | | |
| TITANIUM DIOXIDE & STEARIC | 3 | 3 | — | 5 |
| ACID & ALUMINA & SILICA | | | | |
| TRIMETHYLSILOXYSILICATE | — | — | — | 4 |
| POLYMETHYLSILSESQUIOXANE | — | — | 2 | 2 |
| DIMETHICONE/VINYL | — | — | — | 6 |
| DIMETHICONE CROSSPOLYMER | | | | |
| & ISOCETETH-10 | | | | |
| METHYL METHACRYLATE | — | — | — | 2.5 |
| CROSSPOLYMER | | | | |
| CALCIUM SILICATE | — | — | — | 0.6 |
| POLYSORBATE 85 & AQUA | 4 | 4 | — | — |
| (WATER) | | | | |
| PEG-240/HDI COPOLYMER BIS- | 3.25 | 3.25 | — | — |
| DECYLTETRADECETH-20 ETHER | | | | |
| & AQUA (WATER) & POTASSIUM | | | | |
| LAURATE & TOCOPHEROL | | | | |
| SILICA | 3 | 3 | | |
| ISONONYL ISONONANOATE | 1.75 | 1.75 | — | — |
| HYDROGENATED LECITHIN | 1 | 1 | — | — |
| CETYL ETHYLHEXANOATE | 1 | 1 | — | — |
| DICAPRYLYL CARBONATE | 0.75 | 0.75 | — | — |
| SORBITAN LAURATE | 0.6 | 0.6 | 0.5 | — |
| CI 77891 (TITANIUM DIOXIDE) & | 0.5 | 0.5 | — | — |
| ALUMINUM HYDROXIDE & | | | | |
| STEARIC ACID | | | | |
| VP/EICOSENE COPOLYMER | 0.5 | 0.5 | — | — |
| DIMETHICONE | 0.5 | 0.5 | 3.8 | 7 |
| POTASSIUM SORBATE | 0.45 | 0.45 | — | — |
| CAPRYLYL GLYCOL | 0.4 | 0.4 | — | — |
| HYDROXYETHYL | 0.3 | 0.3 | — | — |
| ACRYLATE/SODIUM | | | | |
| ACRYLOYLDIMETHYL TAURATE | | | | |
| COPOLYMER & SORBITAN | | | | |
| ISOSTEARATE & POLYSORBATE | | | | |
| 60 | | | | |
| TOCOPHERYL ACETATE | 0.2 | 0.2 | 0.2 | — |
| XANTHAN GUM | 0.1 | 0.1 | — | — |
| CITRIC ACID | 0.07 | 0.07 | — | — |
| SODIUM HYALURONATE | 0.05 | 0.05 | — | — |
| SQUALANE | — | — | 6.5 | — |
| HYDROGENATED | — | — | 6 | — |
| POLYISOBUTENE & | | | | |
| DISTEARDIMONIUM HECTORITE | | | | |
| & PROPYLENE CARBONATE | | | | |
| DIMETHICONE & | — | — | 5.5 | — |
| DIMETHICONE/PEG-10/15 | | | | |
| CROSSPOLYMER & | | | | |
| DIPROPYLENE GLYCOL & | | | | |
| TOCOPHEROL | | | | |
| DIMETHICONE & DIMETHICONE | — | — | 5 | — |
| CROSSPOLYMER | | | | |
| PEG-9 | — | — | 2.5 | — |
| POLYDIMETHYLSILOXYETHYL | | | | |
| DIMETHICONE | | | | |
| SYNTHETIC FLUORPHLOGOPITE | — | — | 2 | — |
| ADENOSINE | — | — | 0.04 | — |
| GELLAN GUM | — | 0.1 | — | — |
| AQUA (WATER) | 56.08 | 55.98 | 38.51 | 21.75 |
| SODIUM CHLORIDE | — | — | 1 | 0.5 |
| AQUA (WATER) & | 4 | 4 | 8 | 2 |
| PHENOXYETHANOL & | | | | |
| BIOSACCHARIDE GUM-1 | | | | |
| GLYCERIN | 3 | 3 | 5 | 3 |
| ALCOHOL | 2 | 2 | 4.5 | 5 |
| PARFUM (FRAGRANCE) | 0.4 | 0.4 | 0.2 | 0.35 |
| Preservatives | 0.8 | 0.8 | 0.75 | 0.5 |
| Pigments | 5.5 | 5.5 | 8 | 10.50 |
| TOTAL | 100 | 100 | 100 | 100 |

Formulations 1 to 4 were characterized according to the methods described above. The results of these characterizations are reported in the table below.

TABLE 2
Characterization of compositions 1 to 4

| Compo- | Plateau | Yield | Viscosity | Complex | |
|---|---|---|---|---|---|
| sition | viscosity | stress | 100s−1 | modulus G* | Phase angle |
| no. | (Pa · s) | (Pa) | (Pa · s) | (Pa) | δ (degrees) |
| 1 | 2743 | 25.3 | 1.3 | 314.8 | 8.2 |
| 2 | 3628 | 54.8 | 1.6 | 432.8 | 7.3 |
| 3 | 14,770 | 55.9 | 3.5 | 663.6 | 6.7 |
| 4 | 822 | 1.8 | 1.6 | 29.3 | 6.6 |

Compositions 1 and 2 have a relatively low yield stress, a moderate rigidity reflected by an intermediate complex modulus G*, as well as a moderate elasticity reflected by a sufficiently low δ. These properties afford these two compositions the rheological properties necessary for good stability on transport in the cosmetic article according to the invention, while facilitating the extrusion of the composition through the mesh and adhering to the applicator sponge by virtue of the combination of a low complex modulus G* and a low phase angle δ. Furthermore, the viscosities at 100 s-1 of compositions 1 and 2 are sufficiently low for these compositions to spread easily on the skin in the manner of a fluid composition.

Composition 3 is a gelled composition which has a very high plateau viscosity, as well as a high modulus G* and yield stress. This reflects a significant consistency at rest but insufficient elasticity to allow collection thereof through the mesh of the article according to the invention with an applicator sponge. In addition, the viscosity at 100 s-1 of this composition is high, which is synonymous with poor spreading of composition 3 on the skin.

Composition 4 has a low yield stress, a low complex modulus G*, and a low phase angle δ. This reflects the fact that composition 4 flows spontaneously. Consequently, the composition is not compatible with the cosmetic article according to the invention, because the composition could flow through the mesh, which is not compatible with portable use of the product.

It is understood that the described embodiments are nonlimiting and that it is possible to make improvements to the invention without departing from the scope thereof.

Unless otherwise stated, the word “or” is equivalent to and/or. Similarly, the word “one” is equivalent to “at least one” unless otherwise specified.

1-15. (canceled)

16. A cosmetic article comprising:
(i) a casing,
(ii) a reservoir housed in the casing, comprising a thixotropic cosmetic composition and having an opening for dispensing said cosmetic composition, and
(iii) a mesh covering said opening of the reservoir, configured to be impermeable to the cosmetic composition when the latter is not subject to a shear stress, and permeable to the cosmetic composition when the latter is subject to a shear stress.

17. The cosmetic article as claimed in claim 16, wherein the cosmetic composition comprises an oil-in-water emulsion having an aqueous phase and a fatty phase, the aqueous phase comprising a thixotropic gelling agent.

18. The cosmetic article as claimed in claim 17, wherein the thixotropic gelling agent is a nonionic block copolymer comprising at least one hydrophilic portion and a hydrophobic portion.

19. The cosmetic article as claimed in claim 18, wherein the hydrophilic portions and the hydrophobic portions are connected by at least one urethane bond.

20. The cosmetic article as claimed in claim 18, wherein the nonionic block copolymer is chosen from polyurethanes and derivatives thereof.

21. The cosmetic article as claimed in claim 18, wherein said hydrophilic portion is polyoxyethylene-based and/or wherein said hydrophobic portion is hydrocarbon-based.

22. The cosmetic article as claimed in claim 18, wherein the nonionic block copolymer consists of a chain of three successive blocks, the first and the third block having hydrophilic properties and the second block having hydrophobic properties.

23. The cosmetic article as claimed in claim 17, wherein the content by weight of the thixotropic gelling agent relative to the total weight of the composition is between 0.01% and 20%, preferably between 0.5 and 10%, more preferably still from 1 to 5%.

24. The cosmetic article as claimed in claim 16, wherein the cosmetic composition has a plateau viscosity of greater than or equal to 2500 Pa·s, preferably greater than or equal to 2700 Pa·s.

25. The cosmetic article as claimed in claim 16, wherein the cosmetic composition has a yield stress of less than or equal to 60 Pa, preferably less than or equal to 55 Pa.

26. The cosmetic article as claimed in claim 16, wherein the cosmetic composition has a complex modulus G* of between 200 and 600 Pa, preferably between 300 and 500 Pa.

27. The cosmetic article as claimed in claim 16, wherein the cosmetic composition has a phase angle δ of between 7° and 20°, preferably between 7° and 10°.

28. The cosmetic article as claimed in claim 16, wherein the mesh comprises polyethylene terephthalate fibers and polyurethane fibers.

29. The cosmetic article as claimed in claim 16, wherein the mesh consists of woven knitted fibers.

30. The cosmetic article as claimed in claim 16, further comprising means for ensuring the leaktightness of the reservoir, preferably a cover on which a seal is mounted, preferably a polyethylene terephthalate seal.
